(19) United States
(12) Patent Application Publication
(10) Pub. No.: US 2018/0177147 A1
(43) Pub. Date: Jun. 28, 2018

(54) METHODS AND COMPOSITIONS FOR CULTIVATING PLANTS AND ARTIFICIAL PLANT SEEDS

(71) Applicant: E I DU PONT DE NEMOURS AND COMPANY, Wilmington, DE (US)

(72) Inventors: Timothy Casper, Yorklyn, DE (US); Sharon L. Haynie, Philadelphia, PA (US); Surbhi Mahajan, Grove City, OH (US)

(21) Appl. No.: 15/305,487

(22) PCT Filed: Apr. 24, 2015
(86) PCT No.: PCT/US15/27544

§ 371 Date: Oct. 20, 2016

Related U.S. Application Data
(60) Provisional application No. 61/984,397, filed on Apr. 25, 2014.

(51) Int. Cl. A01H 4/00

(52) U.S. Cl. CPC A01H 4/005; A01H 4/006

(57) ABSTRACT

Methods and compositions for cultivating plants and artificial plant seeds comprising choplets derived from plantlets grown by tissue culture techniques. Methods comprise production of choplets and artificial seeds comprising choplets which are initiated by the micropropagation of plant tissue source material from plants which may be native or transgenic.

### CROSS REFERENCE TO RELATED APPLICATIONS

This application claims the benefit of U.S. Provisional Application No. 61/984397, filed Apr. 25, 2014, which is incorporated by reference in its entirety.

### FIELD

The present disclosure relates generally to methods of cultivating plants and making artificial plant seeds.

### BACKGROUND

Commercial cultivation of plants, such as monocotyledonous and dicotyledonous plants, and others, such as gymnosperms, is of world-wide importance and economic value. The reproduction and sowing, seeding and planting of these organisms is of major importance in the commercial production of plants for agricultural or horticultural purposes. One method of reproduction of plants is micropropagation, a method for the vegetative propagation of plants under conditions which permit rapid multiplication. For the production of new plants, tissue culture methods have been developed for breeding and propagation programs and for the elimination of viruses. Often such techniques, which may produce many new plants, are slow, expensive and require much human handling, which may expose the plants to pathogenic organisms.

Plant regeneration using artificial plant seed technology is an alternative to traditional micropropagation for production and delivery of cloned plantlets, though for many types of plants, this technology remains underdeveloped for large scale commercial use. Much of the work using artificial seed technology has focused on somatic embryos as the tissue of choice. Somatic embryogenesis is often time consuming, labor-intensive, and may lead to genetic or phenotypic changes for many plant species. In some methods, bud tissue was derived from the apical meristem and had low emergence rates when directly transplanted into soil. Thus, there is a need for alternative methods of production of viable plant tissue that can survive transfer to soil and increase the amount of plant tissue available for planting, sowing or seeding.

### BRIEF SUMMARY

The present disclosure comprises methods and compositions for making and using choplets, a form of plant material that develops into an entire plant comprising roots and shoots/leaves under growth-promoting conditions, such as placement in a growth medium. Choplets may be used, for example, for initiating a new crop, for new plantings, or as plant source material in in vitro culture methods. Methods disclosed herein comprise methods for making choplets, using choplets, and methods for making artificial seeds comprising one or more choplets. Compositions disclosed herein comprise choplets, which are a plant material, and compositions comprising artificial seeds comprising one or more choplets.

An aspect of the invention comprises making a choplet, comprising: a) producing at least one plantlet by growing an explant plant tissue in a container for a predetermined time; b) removing at least a portion of the sprouted roots and a portion of the sprouted shoots from at least one plantlet to form at least one choplet; and c) optionally, severing a choplet into smaller sized choplets. A method may comprise, after severing the plantlet to form one or more choplets, placing the one or more choplets in a liquid composition for a predetermined time period.

An aspect of the disclosure comprises a method for cultivating plants comprising a) producing at least one or more choplets from one or more plantlets that are grown from an explant plant tissue in a container for a predetermined time; and b) placing at least one choplet into a structure suitable for planting or placing at least one choplet into a plant growth medium, such as soil. In a method, before placing a choplet into a structure suitable for planting or in soil, a choplet may be placed into a liquid medium for a time period. In an aspect, a choplet may be placed directly in a growth medium, such as potting soil or soil in a field.

An aspect of the disclosure comprises a method of making artificial seeds comprising choplets. A method of making an artificial seed comprises making one or more choplets, and placing one or more choplets in a structure suitable for planting. An aspect of the disclosure further comprises a composition comprising at least one choplet, which is a form of plant material that develops into an entire plant comprising roots and shoots/leaves under growth-promoting conditions, produced by a method disclosed herein. The disclosure further comprises an artificial seed comprising at least one choplet produced by a method disclosed herein and a structure suitable for planting. An artificial seed may further comprise a nutrient source and/or other components to aid in the plant's growth and development. In an aspect, a choplet is made from a plantlet, a form of plant material that has both roots and shoots, and is generally grown from plant tissue under in vitro conditions. For example, a choplet may be made from plant tissue of: a) sugar cane, a graminaceous plant, saccharum spp, saccharum spp hybrids, miscanthus, switchgrass, energycane, sterile grasses, orchids, bamboo, cassava, corn, rice, banana, potato, sweet potato, yam, pineapple, trees, willow, pine trees, poplar, mulberry, ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, citrus, lemon, orange, grapefruit, tangerine, or dayap, b) a genetically modified plant of a), c) a micropropagated version of a), and d) a genetically modified, micropropagated version of a).

### BRIEF DESCRIPTION OF THE FIGURES

FIG. 1. Illustrates an exemplary embodiment of the preparation of a choplet from a plantlet.

FIG. 2. Demonstrates the distribution of plantlet weights after 17 days in tissue culture with varying hardening treatments: Group A: LPM; Group B: final [sucrose]=1.25%; Group C: final [sucrose]=1.25% and [PEG-6000]=0.2%.

FIG. 3. Cone-like lid prepared from plastic microcentrifuge tube.

FIG. 4. Presents certain survival outcomes of choplet seed structures. “Si” are choplet seed structures derived from regenerated plantlets in a sodium silicate hardening medium.

FIG. 5. Presents survival of certain choplets with different shoot lengths.

FIG. 6. A and B are confocal micrographs of A) regenerable plant tissue and B) a choplet.

FIG. 7. A-C are laser ablation tomographs of A) regenerable plant tissue, B) a choplet, and C) a choplet.

### DETAILED DESCRIPTION

The present disclosure comprises methods and compositions for providing plant material, referred to herein as “choplet”, which may be used, for example, for initiating a new crop or for production of new plants. Methods disclosed herein comprise methods for making such plant material, methods for cultivating plants, and methods for making artificial seeds. A choplet plant material is plant tissue capable of growing into a mature plant with the features and genetic identity of the parent plant. Compositions disclosed herein comprise a choplet, compositions useful in making such plant material and compositions comprising artificial seeds comprising one or more choplets. The present disclosure comprises methods for making and using an artificial plant seed comprising the plant tissue material produced by methods disclosed herein.

Plant tissue culture has been used extensively in plant propagation, transformation, mutagenesis, breeding and virus elimination. Such tissue culture systems are generally referred to as micropropagation systems, wherein plant tissue explants are cultured in vitro in a suitable solid or liquid medium, from which mature plants are regenerated. It is well known by a person skilled in the art that conventional micropropagation technology includes micropropagation techniques that relate to propagation and regeneration of plants and plant tissues from an in vitro cultured plant, plant tissue and/or parts thereof.

### I. Methods of Making a Choplet

In one embodiment is disclosed methods of making a choplet, which develops into an entire plant comprising roots and shoots/leaves under growth-promoting conditions, as described herein. A method for making a choplet comprises: a) producing at least one plantlet by growing an explant tissue or a plant tissue suitable for micropropagation techniques in a container for a predetermined time; b) removing at least a portion of the sprouted roots and/or shoots from at least one plantlet to form a choplet; c) optionally, severing the choplet into smaller sized choplets; and d) optionally, placing one or more choplets into a liquid medium. In one embodiment, a method for making a choplet comprises: a) removing at least a portion of both the sprouted roots and the sprouted shoots from a plantlet to form a choplet; and b) optionally, severing that choplet into smaller-sized choplets, wherein a choplet may have a volume of from about 0.1 mm³ to about 125 mm³, and a choplet, when placed in a growth medium, regenerates an entire plant.

Plantlets are made, for example, by methods known to those of skill in the art. Explant tissue, which is tissue removed from a plant and is comprised of cells, or a plant tissue suitable for micropropagation using techniques disclosed herein or known to those of skill in the art, is placed in a medium for culture and subsequently develops into plantlets. Explant tissue may come from a native plant or from a transgenic plant. As used herein, “plantlet” includes reference to young or small plant tissue that has been grown in vitro for a specific amount of time and has sprouted roots and sprouted shoots. Plantlets may be produced asexually by tissue culture or cell culture. Plantlets possess well-differentiated shoots and roots. A plantlet is a plant material used for propagation, as is understood by those of skill in the art. For example, a type of explant tissue may be apical and axillary buds of a plant. Plant apical and axillary buds are small terminal or lateral protuberances on the stem of a vascular plant that may develop into a flower, leaf or shoot. Plant buds arise from meristem tissue and may include overlapping immature leaves or petals.

The source of explant tissue may comprise tissue culture-produced plant material or plant material obtained from field-grown plant materials. A plant tissue source material for making a plantlet may be in vitro cultured regenerable plant tissue. A plant tissue source for making a plantlet may be a choplet. As used herein, “a plant tissue source material” includes explant tissue, in vitro cultured regenerable plant tissue, a choplet serving as plant tissue source material, or other plant tissue suitable for in vitro and micropropagation techniques.

Methods for producing in vitro-cultured whole plantlets are known to those of skill in the art. For example, for the monocot sugarcane, methods of culturing whole plantlets are described in Hendre et al. (1983), Sugar Cane 1:5-8, Cheena and Hussain (2004), Intl. J. Agriculture & Biol. 6(2):257-259, Manickavasagam et al. (2004) Plant Cell Rep. 23: 134-143, and Ali et al. (2008) Pak. J. Bot. 40(1): 139-149, each of which is incorporated by reference herein in its entirety. For example, for sugarcane, apical portions consisting of mature apical and axillary buds of healthy stalks of sugarcane plants can be collected from the field or greenhouse. In some cases, apical and axillary buds containing meristem cells are isolated from the cane stalk, surface-sterilized and inoculated. In vitro-cultured plantlets can be regenerated, proliferated and maintained on medium with or without phytohormones or growth enhancers.

As used herein, “plant” includes references to whole plants, plant organs, plant tissues, seeds and plant cells and progeny of same. Plant cells include, without limitation, cells from choplets, seeds, suspension cultures, embryos, meristematic regions, callus tissue, leaves, roots, shoots, gametophytes, sporophytes, pollen and microspores. The term “plant tissue” includes differentiated and undifferentiated tissues including, but not limited to the following: roots, stems, shoots, leaves, pollen, seeds, tumor tissue and various forms of cells and culture (e.g., single cells, protoplasts, embryos and callus tissue). The plant tissue may be in a plant or in a plant organ, tissue or cell culture.

As used herein, the term “explant tissue” refers to tissue (which comprises cells) taken from any part of and at any stage of development of a mother plant. For example, the source of an explant can be from the leaf, root, shoot, buds, inflorescence, internodes of the plant and/or an explant may comprise mature embryos derived by stressing leaf, shoot, root, buds, inflorescence and/or internodes of the plant. For example, stressing may be achieved by treating the plant part with 95% ethanol for a period of time (preferably 1-5 hours) and/or cooling the plant part to a temperature of approximately 5-15° C. for approximately 1-3 months. In an aspect, explant tissue can be from a bud. In an aspect, roots and shoots are used as the explant material.

In an aspect, the disclosure relates to a method of making one or more choplets wherein a plant tissue source material is grown in a container. In an aspect, the container is a bioreactor. In an aspect, the container is a plantform. In an aspect, the container is a flask. In an aspect, the container is an array.

In a method of making choplets, a plant tissue source material may be grown in a container for a period of about 1 to about 30 days to form one or more plantlets. In an aspect, a plant tissue source material can be grown for about 8 to about 25 days to form one or more plantlets. In an aspect, a plant tissue source material can be grown for about 10 days to form plantlets. In an aspect, a plant tissue source material can be grown for about 14 days to form plantlets. In an aspect, a plant tissue source material can be grown for about 17 days to form plantlets. In an aspect, a plant tissue source material can be grown for about 20 days to form plantlets.

In an aspect, a plant tissue source material is grown to form plantlets in a composition that comprises a growth medium, which may be a liquid nutrient solution. In an aspect, one or more choplets may be used to form one or more plantlets in vitro or in vivo. For example, a choplet may be made from plant tissue of: a) sugar cane, a graminaceous plant, saccharum spp, saccharum spp hybrids, miscanthus, switchgrass, energycane, sterile grasses, orchids, bamboo, cassava, corn, rice, banana, potato, sweet potato, yam, pineapple, trees, willow, pine trees, poplar, mulberry, ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, citrus, lemon, orange, grapefruit, tangerine, or dayap, b) a genetically modified plant of a), c) a micropropagated version of a), and d) a genetically modified, micropropagated version of a).

In one embodiment of a method for making choplets, in vitro culture conditions for plantlets are maintained for 10 days. At this time, the plantlets are removed and choplets are formed from the 10 day plantlets. In another embodiment, the culture conditions are maintained for at least 10 days to about two weeks or to about three weeks, the plantlets are removed at that time, and choplets are formed from the plantlets so grown. Genera of plants may have differing growing periods to produce a plantlet that has both sprouted roots and sprouted shoots, such that the plantlet is developed sufficiently that it can be used to form a choplet, and those of skill in the art can determine the predetermined growth period for such a plantlet of a particular type of plant.

A method of making choplets may comprise removing at least a portion of the length of the sprouted roots and sprouted shoots from at least one plantlet to form a choplet. For example, the sprouted roots of a plantlet extend outwardly from the base of the plantlet and the sprouted shoots extend outwardly generally from the base of the plantlet in a direction opposite from the roots. The sprouted roots and sprouted shoots may or may not be of a similar length extending from the base of the plantlet. The lengths of the sprouted roots and shoots are removed from the plantlet by severing, slicing, or otherwise cutting the plantlet to remove at least a portion of the lengths of the roots and shoots from a plantlet to form a choplet. The removal of the shoots and roots may be performed manually, such as by hand cutting, or by an automated device, and may comprise use of a knife, an automated or semi-automated knife, laser, wire, scissors or other cutting implements known to those of skill in the art. In an aspect, the majority of the lengths of the sprouted roots and shoots are removed from the plantlet. In an aspect, the length of sprouted root material removed may be more than the length of sprouted shoot material removed. In an aspect, the amount of sprouted shoot material removed may be more than the length of sprouted root material removed. In an aspect, substantially all of the length of sprouted root and/or shoot material may be removed. In an aspect, only a small portion of the length of the sprouted root and/or shoot material may be removed from a plantlet. For example, removed from the plantlet is 0.1 cm of shoot and/or root material length; removed is 0.5 cm of shoot and/or root material length; removed is 0.7 cm of shoot and/or root material length; removed is 0.9 cm of shoot and/or root material length; removed is 1 cm of shoot and/or root material length; removed is 2 cm of shoot and/or root material length; removed is 3 cm of shoot and/or root material length; removed is 4 cm of shoot and/or root material length; removed is 5 cm of shoot and/or root material length; removed is 6 cm of shoot and/or root material length; removed is 7 cm of shoot and/or root material length; removed is 8 cm of shoot and/or root material length; removed is 9 cm of shoot and/or root material length; removed is 10 cm of shoot and/or root material length; removed is 11 cm of shoot and/or root material length; removed is 12 cm of shoot and/or root material length; removed is 13 cm of shoot and/or root material length; removed is 14 cm of shoot and/or root material length; removed is 15 cm of shoot and/or root material length; removed is 16 cm of shoot and/or root material length; removed is 17 cm of shoot and/or root material length; removed is 18 cm of shoot and/or root material length; removed is 19 cm of shoot and/or root material length; removed is 20 cm of shoot and/or root material length; or removed is more than 20 cm of shoot and/or root material length from a plantlet.

A choplet refers to the remainder of the plantlet after removal of the desired length of shoots and roots, or a choplet may be formed by apportioning the remainder of the plantlet after removal of the desired length of shoots and roots (choplet) into smaller volume choplets. As used herein, the term “choplet” may refer to small pieces, fragments or portions of the remainder of the plantlet after removal of the desired length of shoots and roots and may be produced by severing, slicing, or otherwise cutting the original choplet into smaller pieces of tissue, for example, into cubes, each of which is still capable of developing into an entire plant having roots and shoots. The division of a choplet may be performed manually, such as by hand cutting the base material, or by an automated device, and may comprise use of a knife, an automated or semi-automated knife, laser, wire, scissors or other cutting implements known to those of skill in the art. The term “choplet” may refer to a larger- or smaller-sized plant material.

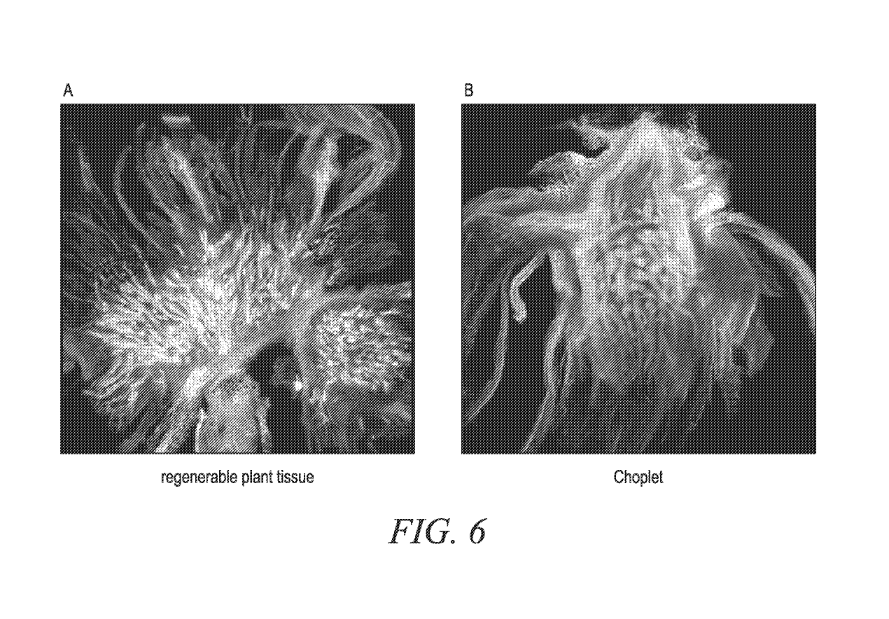

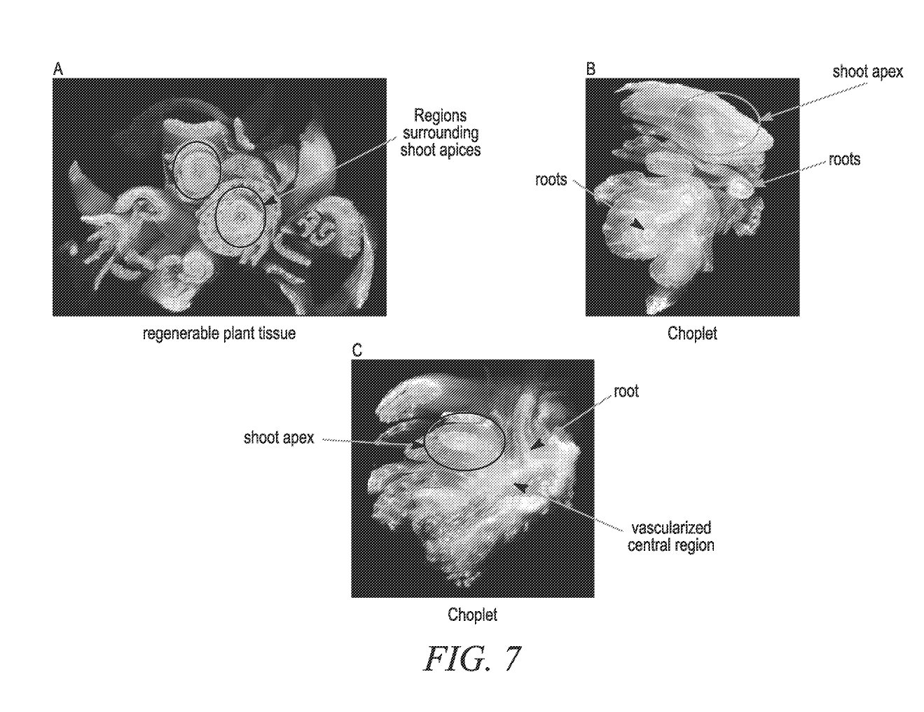

A “choplet” is plant material that results from the removal of a portion of the length of shoots and roots of a plantlet, wherein the plantlet has been grown from a plant tissue source material in ex vivo culture for a time period of from about 10 to about 25 days, and wherein the choplet has both a shoot apical meristem-like activity zone and a root apical meristem-like activity zone. A choplet must contain both root plant material and shoot plant material. A choplet has both shoot and root meristem-like activity properties, but the actual root and shoot tissues are not yet organized tissue structures of roots and shoots. The root and shoot cells have root and shoot apical meristem-like activity, but are not structured like apical meristem. For example, see FIGS. 6 and 7. A choplet is capable of developing into a complete plant having both roots and shoots or leaves. A choplet comprises cells having the properties of cell division, differentiation and a predisposition to form a root or a shoot, under normal growth conditions.

A choplet is defined as having a shoot apical meristem-like activity zone that is not too far in distance from the root apical meristem-like activity zone, and if both of these zones are not present a plant will not develop from the choplet, and if no plant can develop, the plant material is not a choplet. A choplet is plant material derived from a plantlet in which the shoot apical meristem-like activity zone is within about 0.01 mm to about 20 mm of the root apical meristem-like activity zone, or about 0.02 mm to about 20 mm, about 0.03 mm to about 20 mm, about 0.05 mm to about 20 mm, about 0.07 mm to about 20 mm. about 0.09 mm to about 20 mm, about 1.0 mm to about 20 mm, about 2.0 mm to about 20 mm, about 3.0 mm to about 20 mm, about 4.0 mm to about 20 mm, about 5.0 mm to about 20 mm, about 6.0 mm to about 20 mm, about 7.0 mm to about 20 mm, about 8.0 mm to about 20 mm, about 9.0 mm to about 20 mm, about 10 mm to about 20 mm, about 11 mm to about 20 mm, about 12 mm to about 20 mm, about 13 mm to about 20 mm, about 14 mm to about 20 mm, about 15 mm to about 20 mm, about 16 mm to about 20 mm, about 17 mm to about 20 mm, about 18 mm to about 20 mm, about 19 mm to about 20 mm, wherein the shoot apical meristem-like activity zone is within about 0.01 to about 0.03 mm of the root apical meristem-like activity zone, or within about 0.01 to about 0.05 mm, within about 0.01 mm to about 0.07 mm, within about 0.01 mm to about 0.09 mm, within about 0.1 mm to about 1.0 mm, within about 0.1 mm to about 2 mm, within about 0.1 mm to about 3 mm, within about 0.1 mm to about 4 mm, within about 0.1 mm to about 5 mm, within about 0.1 mm to about 6 mm, within about 0.1 mm to about 7 mm, within about 0.1 mm to about 8 mm, within about 0.1 mm to about 9 mm, within about 0.1 mm to about 10 mm, within about 0.1 mm to about 11 mm, within about 0.1 mm to about 12 mm, within about 0.1 mm to about 13 mm, within about 0.1 mm to about 14 mm, within about 0.1 mm to about 15 mm, within about 0.1 mm to about 16 mm, within about 0.1 mm to about 17 mm, within about 0.1 mm to about 18 mm, within about 0.1 mm to about 19 mm, within about 0.1 mm to about 20 mm. Those of skill in the art are capable of determining meristematic-like activity and cellular types associated with roots and shoots using techniques known in the art.

Plantlets may be reduced in size to form one or more choplets by first severing a portion of the lengths of the sprouted root and shoot material. In some aspects, 0.1-5 cm lengths of the sprouted shoot and root material may be retained. A particular length of shoot and/or root material may remain on a choplet. A choplet may retain about 0.1 cm of shoot and/or root material with the remainder of the shoot and/or root material severed, retained is 0.5 cm of shoot and/or root material, retained is 0.7 cm of shoot and/or root material, retained is 0.9 cm of shoot and/or root material, retained is 1 cm of shoot and/or root material, retained is 2 cm of shoot and/or root material, retained is 3 cm of shoot and/or root material, retained is 4 cm of shoot and/or root material, retained is 5 cm of shoot and/or root material, retained is 6 cm of shoot and/or root material, retained is 7 cm of shoot and/or root material, retained is 8 cm of shoot and/or root material, retained is 9 cm of shoot and/or root material, retained is 10 cm of shoot and/or root material, retained is 11 cm of shoot and/or root material, retained is 12 cm of shoot and/or root material, retained is 13 cm of shoot and/or root material, retained is 14 cm of shoot and/or root material, retained is 15 cm of shoot and/or root material, retained is 16 cm of shoot and/or root material, retained is 17 cm of shoot and/or root material, retained is 18 cm of shoot and/or root material, retained is 19 cm of shoot and/or root material, retained is 20 cm of shoot and/or root material, or retained is more than 20 cm of shoot and/or root material of a plantlet.

To maintain the spatial relationship of the shoot apical meristem-like activity zone to the root apical meristem-like activity zone in a choplet, severing a choplet along a perpendicular axis through both meristem-like zones can be used to produce multiple smaller sized choplets from a larger sized choplet.

The term “meristem-like activity” refers to plant cells capable of cell division and characterized by a commitment by the section of cells that will develop into roots or shoots. Similar activity is found at growing points or tissues in plants such as root tips, stem apices, lateral buds, or the like. Choplet meristematic-like activity is different from meristematic tissue in that meristematic tissue exhibits undifferentiated growth and capacity for organ differentiation and totipotency. Thus, a single transformed meristematic cell could be recovered as a whole transformed plant. Such meristem tissue includes the cells found in the cambium or growing points capable of further development.

Regenerable plant tissue is plant tissue with meristematic properties capable of initiating shoot formation, proliferation of multiple meristem initials, resulting in an increase in plant tissue mass. Regenerable plant tissue can lead to plantlet formation when cultured in the absence of plant hormones, such as cytokinin.

As shown in FIGS. 6A and B and 7A-C, a choplet is anatomically distinct from regenerable plant tissue, as defined in co-pending U.S. Provisional Patent Application Ser. No. 61/984093, filed Apr. 25, 2014, entitled Sugarcane Process, which is herein incorporated in its entirety. A choplet has reduced trichosomes compared to regenerable plant tissue. A choplet has roots derived from a central region of the choplet, whereas regenerable plant tissue has no roots as it is maintained on a media that suppresses root development. The vasculature of regenerable plant tissue is relatively parallel (See FIG. 6A), whereas vasculature of a choplet (FIG. 6B) is not relatively parallel. For example, regenerable plant tissue may be obtained by: a) identifying and excising meristematic tissue from plants; b) in a first phase, growing the meristematic tissue on solid support media comprising plant hormones to suppress root development; and c) in a second phase, growing the tissue from step b) in liquid media comprising paclo-type compounds and/or plant hormones including hormones to suppress root development, and allowing the tissue to proliferate to a larger mass to form regenerable plant tissue.

In an aspect, the present disclosure provides a method of making choplets comprising severing at least one plantlet to form one or more choplets, wherein a choplet has a particular volume. A composition resulting from a method of making choplets may comprise choplets. In an aspect, a composition resulting from a method of making choplets may comprise about 50% or more of choplets, about 60% or more of choplets, about 70% or more of choplets, about 75% or more of choplets, about 80% or more of choplets, about 90% or more of choplets, about 95% or more of choplets, about 96% or more of choplets, about 97% or more of choplets, about 98% or more of choplets, about 99% or more of choplets, or 100% of choplets.

In an aspect, the choplet may be formed with no particular volume restrictions as long as the choplet comprises shoot apical meristem-like activity zone in spatial relationship to the root apical meristem-like activity zone. In an aspect, a choplet is generally cube-shaped and may have a volume of from about 0.5 mm³ to about 125 mm³. One side of a cube-shaped choplet may be from about 0.01 mm, about 0.02 mm, about 0.03 mm, about 0.04 mm, about 0.05 mm, from about 0.06 mm, about 0.07 mm, about 0.08 mm, about 0.09 mm, about 1.0 mm, about 1.2 mm, about 1.4 mm, about 1.5 mm, about 1.7 mm, about 1.9 mm, about 2.0 mm, about 2.2 mm, about 2.4 mm, about 2.6 mm, about 2.8 mm, about 3.0 mm, about 3.2 mm, about 3.4 mm, about 3.6 mm, about 3.8 mm, about 4.0 mm, about 4.2 mm, about 4.4 mm, about 4.6 mm, about 4.8 mm, up to about 5.0 mm, in length. In an aspect, a plurality of choplets may have substantially uniform sized sides and volume. In an aspect, a plurality of choplets may have substantially non-uniform sized sides and volume.

After removing a portion of the root and shoot length or after apportioning the choplet formed after removal of the root and root length into smaller volume choplets, a choplet may be placed in a liquid medium. One embodiment comprises making a choplet and forming one or more choplets before: placing a choplet into a structure, such as an artificial seed structure; planting the choplet; or placing one or more choplets into a liquid medium composition for a predetermined time period.

In a particular aspect, one or more choplets may reside in the liquid medium composition for a time period of about 30 minutes to about 3 days, or longer. A range of exposure to a liquid medium, such as water or a suitable liquid plant nutritive medium, may include from about 5-30 minutes, from about 10-30 minutes, from about 20-30 minutes, from about 30 minutes to one hour, from about 45 minutes to one hour, from about 50 minutes to about one hour, from about 30 minutes to about 2 hours, from about 75 minutes to about 2 hours, from about 90 minutes to about 2 hours, from about 2 hours to about 2.5 hours, from about 10 minutes to about 3 hours, from about 10 minutes to about 4 hours, from about 10 minutes to about 5 hours, from about 10 minutes to about 6 hours, from about 10 minutes to about 7 hours, from about 10 minutes to about 8 hours, from about 10 minutes to about 9 hours, from about 10 minutes to about 10 hours, from about 10 minutes to about 11 hours, from about 10 minutes to about 12 hours, from about 10 minutes to about 15 hours, from about 10 minutes to about 20 hours, from about 10 minutes to about 2 days, from about 10 minutes to about 3 days, from about 10 minutes to about 4 days, from about 1 day to about 2 days, from about 1 day to about 3 days, from about 1 day to about 4 days, from about 1 day to about 5 days, from about 1 day to about 6 days, or longer as can be determined by one of skill in the art. A liquid medium can comprise water or a nutritive media known to those of skill in the art

### II. Methods of Making Artificial Seeds

The disclosure further comprises artificial seeds and methods of making and using artificial seeds. An artificial seed may be made by a method comprising: a) producing at least one choplet from a plantlet grown for a predetermined time in in vitro culture conditions from a plant tissue source material; and b) placing at least one choplet into a structure suitable for planting to form an artificial seed comprising a choplet.

As used herein, a structure suitable for planting comprises an “artificial plant seed” which is an object that is human-made, which comprises one or more choplets, and which may comprise components necessary to facilitate plant growth, and from which a plant may grow and be established from its own plant tissue, but wherein the plant tissue is not typically the same as the plant's natural seed. By contrast, a natural seed is produced by plants in a natural biological process without human intervention. An artificial plant seed is able to regenerate into a plant and that plant may undergo germination. The terms “artificial plant seed” and “artificial seed” may be used interchangeably herein.

The plant material of an artificial seed of the present invention comprises one or more choplets as disclosed herein and produced by methods disclosed herein. An artificial seed may comprise other plant materials, growth medium and/or other growth promoting and enhancing components known to those of skill in the art for aiding and enhancing plant growth and establishment. In one embodiment, an artificial seed comprises at least one choplet.

A composition comprising one or more artificial seeds may comprise a growth medium composition in which one or more artificial seeds have been planted. For example, potting soil or soil in a field in which artificial seeds comprising choplets have been planted comprises a composition comprising one or more artificial seeds.

In an aspect, the disclosure provides a method of making artificial seeds comprising placing at least one choplet into a structure suitable for planting. A structure suitable for planting may be an artificial seed. In an aspect a structure suitable for planting may comprise plastic, paper, wood, clay, or any other material. The shape of a structure may be tubular, spherical, cubical, conic, or any other shape suitable for planting. For example, in an aspect, the structure suitable for planting can be a paper tube. In an aspect, the structure can be a plastic tube. In an aspect, the structure may be sealed on at least one end. In an aspect, a tube may be sealed on at least one end. In an aspect, a structure suitable for planting is a paper tube with a cover. The cover may comprise a bilayer film, a triblock film, a parafilm, and/or any combination thereof. Artificial seed structures are known to those of skill in the art, for example International Patent Application No. PCT/US2012/070766 (International Publication No. WO 2013/096531), which is herein incorporated in its entirety. For example, an artificial seed may comprise one or more choplets, a container comprising a degradable portion, an unobstructed airspace, and a nutrient source, and further comprising one or more features including, but not limited to: a penetrable or degradable region through which one or more choplets grows; a monolayer water soluble portion of the container; a region of the container that flows or creeps between about 1° C. and 50° C.; a separable closure which is physically displaced during choplet growth; one or more openings in sides or bottom of the container; a conical or tapered region leading to an opening less than 2 cm wide at the apex and wherein the angle of the conical or tapered region is less than 135 degrees measured from opposite sides; and a plurality of flexible flaps through which the choplet grows.

In an embodiment, the container of an artificial seed may comprise a component such as: a) a cylindrical tube with a conical top; b) a two part tube with a porous bottom section and a nonporous top section; c) a flexible packet; d) a semi-flexible packet; e) a rolled tube structure, capable of unraveling; f) an anchoring device; g) a multi-part tube with a hinged edge; h) a multi-part tube held together with adhesive; i) a tubular shape; j) a container portion in contact with soil that degrades faster than the portion above soil; k) an airspace comprising multiple compartments; l) a closed bottom end that retains moisture; m) a cap attached by an adhesive joint; n) a cap attached by insertion into the container; or o) a weak region.

In an embodiment, the container of an artificial seed or a closure may comprise a material such as: a) a transparent, translucent or semi-translucent material; b) an opaque material; c) a porous material; d) a nonporous material; e) a permeable material; f) an impermeable material; or g) any one of materials a) through f), wherein the material is biodegradable, hydrolytically degradable, or compostable.

In an embodiment, one or more of openings may be secured using a component selected from the group consisting of: a) a crimp; b) a fold; c) a porous material; d) mesh; e) screen; f) cotton; g) gauze; and, h) a staple.

In an embodiment, an artificial seed may comprise an agent such as: a) a fungicide; b) a nematicide; c) an insecticide; d) an antimicrobial compound; e) an antibiotic; f) a biocide; g) an herbicide; h) plant growth regulator or stimulator; i) microbes; j) a molluscicide; k) a miticide; l) an acaricide; m) a bird repellent; n) an insect repellent; o) a plant hormone; or p) a rodent repellent.

An artificial seed of the present invention may comprise a capsule comprising one or more choplets. For example, capsules such as those made by Morishita Jintan Co., Ltd., of Japan are contemplated by the present invention. For example, see EP Patent No. 0525731 B1, U.S. Pat. No. 4,695,466, U.S. Pat. No. 6,982,095, U.S. Patent Application Publication No. 20040175412, US Patent Application Publication No. 20050283849, and International Patent Application Publication No. WO2003001927A1, each of which is hereby incorporated by reference in its entirety.

A structure suitable for planting such as an artificial seed may be made of materials known to those of skill in the art. For example, a structure suitable for planting and/or one or more closures of the structure may be made from or comprise one or more of polyesters, polyamides, polyolefins, cellulose, cellulose derivatives, polysaccharides, polyethers, polyurethanes, polycarbonates, poly(alkyl methacrylate)s, poly(alkyl acrylate)s, poly(acrylic acids), poly(meth)acrylic acids, polyphosphazenes, polyimides, polyanhydrides, polyamines, polydienes, polyacrylamides, poly(siloxanes), poly(vinyl alcohol), poly(vinyl esters), poly(vinyl ethers), natural polymers, block copolymers, crosslinked polymers, proteins, waxes, oils, greases, water soluble polymers, poly(ethylene glycol), salts of poly(acrylic acid), poly(vinyl alcohol), plasticizers, antioxidants, nucleating agents, impact modifiers, processing aids, tougheners, colorants, fillers, stabilizers, flame retardants, natural rubber, polysulfones, or polysulfides; or blends thereof; or crosslinked versions thereof. For example, a structure suitable for planting may be made from or comprise one or more of a) amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, or cellulose butyrate acetate, b) a polyester with greater than 5 mol percent aliphatic monomer content, c) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), (poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content, d) a plasticizer, wherein the plasticizer is present at less than 30 wt % of the total composition, e) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water, f) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, dimethyl siloxane, succinic anhydride, a diisocyanate, a crosslinker, or phthalic anhydride, g) an antioxidant, a nucleating agent, an impact modifier, a processing aid, a toughener, a colorant, a filler, a stabilizer, or a flame retardant, h) paper, water soluble paper, recycled paper, bond paper, kraft paper, waxed paper, or coated paper. For example the structure suitable for planting and/or one or more covers of the structure may be made from or comprise one or more of a) random, block or gradient copolymers of lactic acid with caprolactone, b) random, block or gradient copolymers of lactic acid with dimethylsiloxane, c) an alkyd resin, d) poly(vinyl alcohol), poly(acrylamide), poly(vinyl pyrrolidone), starch, cellulose, glycerol, poly(ethylene glycol), citric acid, urea, water, sodium acetate, potassium nitrate, ammonium nitrate, fertilizers, agar, xanthan gum, alginate, hydroxypropylcellulose, methylcellulose, carboxymethylcellulose, guar gum, pectin, a water soluble protein, a water soluble carbohydrate, a water soluble synthetic polymer, gelatin, or sodium carboxymethylcellulose, and crosslinked versions thereof, e) blends of two or more of the following: poly(vinyl alcohol), starch, cellulose, glycerol, poly(ethylene glycol), poly(acrylamide), poly(vinyl pyrrolidone), citric acid, urea, water, sodium acetate, potassium nitrate, ammonium nitrate, fertilizers, agar, xanthan gum, alginate, hydroxypropylcellulose, methylcellulose, carboxymethylcellulose, a water soluble protein, a water soluble carbohydrate, a water soluble synthetic polymer, gelatin, a crosslinker, or sodium carboxymethylcellulose, f) a gel comprising a block copolymer and an oil, g) sodium carboxymethylcellulose, h) wax-impregnated water soluble paper, i) amorphous poly(D,L-lactic acid), poly(lactic acid), poly(L-lactic acid), poly(D-lactic acid), poly(meso-lactic acid), poly(rac-lactic acid), or poly(D,L-lactic acid), poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, thermoplastic starch, gelatin, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate; or a crosslinked version thereof, j) a polyester with greater than 5 mol percent aliphatic monomer content, k) a crosslinked version of amorphous poly(D,L-lactic acid), poly(lactic acid), poly(L-lactic acid), poly(D-lactic acid), poly(meso-lactic acid), poly(rac-lactic acid), or poly(D,L-lactic acid), poly(hydroxyalkanoate), poly(hydroxybutyrate), poly(hydroxybutyrate-co-valerate), poly(caprolactone), poly(butylene succinate), poly(ethylene succinate), poly(ethylene carbonate), poly(propylene carbonate), starch, gelatin, thermoplastic starch, poly(butylene terephthalate adipate), poly(propylene terephthalate succinate), poly(propylene terephthalate adipate), poly(vinyl alcohol), poly(ethylene glycol), cellulose, chitosan, cellulose acetate, cellulose butyrate acetate, or a polyester with greater than 5 mol percent aliphatic monomer content, l) a plasticizer, m) acetyl tributyl citrate, tributyl citrate, di-n-octyl sebacate, di-2-ethylhexylsebacate, di-2-ethylhexylsuccinate, diisooctyl adipate, di-2-ethylhexyl adipate, diisooctyl glutarate, di-2-ethylhexyl glutarate, poly(ethylene glycol), poly(ethylene glycol) monolaurate, sorbitol, glycerol, poly(propylene glycol), or water, n) copolymers of two or more of caprolactone, lactic acid, D-lactide, L-lactide, meso-lactide, D,L-lactide, sebacic acid, succinic acid, adipic acid, glycolic acid, oxalic acid, ethylene glycol, 1,2-propanediol, 1,3-propanediol, 1,3-butanediol, 1,4-butanediol, 1,5-pentanediol, 2,2,4,4-tetramethyl-1,3-cyclobutanediol, 1,6-hexanediol, terephthalic acid, isophthalic acid, succinic anhydride, a diisocyanate, a crosslinker, or phthalic anhydride, o) an antioxidant, a nucleating agent, an impact modifier, a processing aid, a toughener, a colorant, a filler, a stabilizer, or a flame retardant, p) a wax, Parafilm® or Nescofilm®, a hydrophobic substance, a fat, a triglyceride, fatty acid, fatty alcohol, a lipid, an oil, polyethylene, polypropylene, ethylene propylene copolymers, polybutadiene, polyisoprene, polyisobutylene, polyolefin oligomers, and crosslinked versions or blends thereof.

In an aspect, a structure suitable for planting comprises a growth medium. The growth medium may be any growth medium known to those of skill in the art and may include a natural or synthetic substrate or a mixture thereof. Non-limiting examples include soil, clay, sand, silt, small wood chops, cellulose, decayed organic residues, vermiculite, coconut fibers and the like and combinations thereof. The growth medium may also be a nutrient solution, such as an aqueous solution. A growth medium may contain growth factors, fertilizers, buffers, ion exchangers, inorganic salts, such as calcium salts (e.g. calcium nitrate, calcium sulphate, calcium hydrogen phosphate) magnesium salts (e.g. magnesium nitrate, magnesium sulphate), potassium salts (e.g. potassium dihydrogen phosphate, potassium nitrate), iron salts (e.g. ferrous sulfate, ferric chloride) and micronutrients (e.g. lithium salts, such as lithium chloride, copper salts, such as copper sulfate, zinc salts, such as zinc sulfate, aluminum salts, such as aluminum sulfate, nickel salts, such as nickel sulfate, tin salts, such as tin chloride, cobalt salts, such as cobalt nitrate, boric acid) and combinations thereof such as Knop's nutrition solution and Hoagland's A-Z solution, and the like. The nutrient solution may be form-stabilized, e.g. by an inorganic substrate, such as expanded clay. In an aspect, the growth medium may be the soil in a field. The growth medium may be treated with agents known to aid in plant growth and may comprise other agents, such as growth promoters, fertilizers, etc., known to those of skill in the art.

In an aspect, the growth medium may be treated before, during and/or after planting one or more choplets in a structure to form an artificial seed with at least one fertilizer, fungicide, insecticide, nematicide, growth regulator, superabsorber, and/or growth-promoting bacteria and/or combinations thereof. In an aspect, the growth medium can be treated with at least one fungicide, and/or at least one growth regulator. In an aspect, a growth medium may be treated before, during and/or after planting the artificial seed as disclosed herein.

In an aspect, a structure suitable for planting comprises a closure. A closure may be provided with ventilation holes in it. Such ventilation holes may be closeable or not. A closure may comprise a micro-mesh screen, microporous membrane, or any material which allows the flow of air between the interior and exterior of the structure, and which may or may not prevent bacteria, spores, and the like from entering the structure. A closure, such as a micro-mesh screen or the like may or may not allow the evaporation of moisture from the structure and, therefore, may aid in regulating the humidity within the structure. In an aspect, a closure comprises a transparent material so as to allow light to be admitted.

In an aspect, one or more choplets are placed into a structure suitable for planting to form an artificial seed. The artificial seed may be stored, packaged with other seeds, and/or planted within a growth medium, for example, by being placed within soil in a field or greenhouse. New plants grow from the choplets.

### III. Methods of Cultivating a Plant or Regenerating a Plant

In an aspect, the present invention discloses methods of cultivating plants or regenerating plants from artificial seeds disclosed herein, or from choplets, produced as described herein. A method for cultivating or regenerating a plant comprises placing at least one artificial seed comprising at least one choplet into a growth medium under conditions conducive to plant growth. A method for cultivating or regenerating a plant comprises placing at least one choplet into a growth medium under conditions conducive to plant growth. Conditions for plant growth include, but are not limited to, those related to outdoor, greenhouse, nursery, field, artificial, tissue culture, bioreactors, or micropropagation conditions known to those of skill in the art. Biological factors including, but not limited to, temperature, light, moisture, growth enhancement, and fertilizer, may or may not be controlled.

In an aspect, the present invention comprises a method of cultivating plants comprising placing at least one choplet directly into a growth medium, which for example may be in soil in field or a greenhouse, or a growth medium contained within a container. A choplet may be placed in in vitro culture conditions for development into a plantlet. A plantlet derived from a choplet may be used to make one or more choplets.

In an aspect, the present invention comprises a method of making artificial seeds comprising placing at least one choplet into a structure suitable for planting, i.e., providing an artificial seed. A method for producing plants comprises planting one or a plurality of artificial seeds in a growth medium, which for example may be soil in field or a greenhouse, or a growth medium contained within a container. A container of growth medium may be made of a conventional material or a biodegradable material. The container of growth medium may be, for example, plastic, paper, wood, clay, or any other material that can contain growth medium. Biodegradable containers of growth medium into which artificial seeds of the present invention have been planted can be provided directly to a field. A biodegradable structure can be made from a biodegradable material which may be made in part or in whole from biodegradable polymers including, but not limited to, starch, cellulose, cellulosic material, polylactic acid, caoutchouc, paper, paperboard, pulp of cellulosic origin, straw, bagasse, sawdust, natural fibers, and/or combinations thereof, or other polymers and compositions disclosed herein.

The temperature at which new plants grow from choplets may be dependent on the type of plant of which the choplet was originally derived. For example, a new plant may grow from a choplet at a temperature of at least 10° C. In an aspect, the new plants are grown from the choplets at a temperature from the range of 10° C. to 35° C. In another aspect, the new plants are grown from the choplets at a temperature from the range of 15° C. to 35° C. In an aspect, the new plants are grown from the choplets at a temperature from the range of 18° C. to 35° C. In an aspect, new plants are grown from the choplets at a temperature from the range of 20° C. to 35° C. In an aspect, the new plants are grown from the choplets at a temperature from the range of 22° C. to 35° C. In an aspect, the new plants are grown from the choplets at a temperature from the range of 25° C. to 35° C., e.g. 25° C. to 32° C., 25° C. to 30° C., 25° C. to 28° C., 25° C. to 26° C.

In one aspect, new plants are grown from the choplets at a humidity ranging from 40 to 100%. In an aspect, new plants are grown from the choplets at a humidity ranging from 50 to 95%. In an aspect, the new plants are grown from the choplets at a humidity ranging from 70 to 90%. In an aspect, the new plants are grown from the choplets at a humidity ranging from 70 to 80%.

In an aspect, the choplets are placed into a structure suitable for planting, wherein the structure is then planted into a container comprising a growth medium. In an aspect, the choplets are planted directly into a container comprising a growth medium. Such a container of growth medium may be covered with one or more covering materials. Covering materials may include, for example, textile mats and cover foils customarily used for thermal insulation and/or protection in agriculture, such as agriculture plastic foil, preferably black foil, for example in the form of foil tunnels, or fleece mats.

Placing the choplets into the structure suitable for planting or directly into a container of growth medium can be accomplished manually, semi-automatically, or in an automated fashion.

Growth conditions for plantlets and/or for artificial seeds, and plants regenerated from choplets produced as described herein may comprise agents, such as cytokines, growth promoting compounds, fertilizers, super-absorbers, growth-promoting microorganisms, antimicrobial compounds, fungicides, insecticides, nematocides, biological control agents, and other active compounds.

Examples of fungicides include, but are not limited to:

A) azoles, including azaconazole, bitertanol, bromuconazole, cyproconazole, difenoconazole, diniconazole, diniconazole-M, epoxiconazole, etaconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, tradimefon, triadimenol, triticonazole, uniconazole, 1-(4-chloro-phenyl)-2-([1,2,4]triazol-1-yl)-cycloheptanol, cyazofamid, imazalil, pefurazoate, prochloraz, triflumizol, benomyl, carbendazim, fuberidazole, thiabendazole, ethaboxam, etridiazole, hymexazole and 2-(4-chloro-phenyl)-N-[4-(3,4-dimethoxy-phenyl)-isoxazol-5-yl]-2-prop-2-ynyloxy-acetamide;

B) strobilurins, including azoxystrobin, dimoxystrobin, enestroburin, fluoxastrobin, kresoxim-methyl, metominostrobin, orysastrobin, picoxystrobin, pyraclostrobin, pyraoxystrobin, pyrametostrobin, pyribencarb, trifloxystrobin, 2-(2-(6-(3-chloro-2-methyl-phen-oxy)-5-fluoro-pyrimidin-4-yloxy)-phenyl)-2-methoxyimino-N-methyl-acetamide, 3-methoxy-2-(2-(N-(4-methoxy-phenyl)-cyclopropane-carboximidoylsulfanylmethyl)-phenyl)-acrylic acid methyl ester, methyl (2-chloro-5-[1-(3-methylbenzyloxyimino)-ethyl]benzyl)carbamate and 2-(2-(3-(2,6-dichlorophenyl)-1-methyl-allylidene-aminooxymethyl)-phenyl)-2-methoxyimino-N-methyl-acetamide;

C) carboxamides, including benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penthiopyrad, sedaxane, tecloftalam, thifluzamide, tiadinil, 2-amino-4-methyl-thiazole-5-carboxanilide, 2-chloro-N-(1,1,3-trimethyl-indan-4-yl)-nicotinamide, N-(3′,4′,5′-trifluorobiphenyl-2-yl)-3-difluoromethyl-1-methyl-1H-pyrazole-4-carboxamide (fluxapyroxade), N-(4′-trifluoromethylthiobiphenyl-2-yl)-3-difluoromethyl-1-methyl-1H-pyrazole-4-carboxamide, N-(2-(1,3-dimethyl-butyl)-phenyl)-1,3-dimethyl-5-fluoro-1H pyrazole-4-carboxamide and N-(2-(1,3,3-trimethyl-butyl)-phenyl)-1,3-dimethyl-5-fluoro-1H-pyrazole-4-carboxamide, dimethomorph, flumorph, pyrimorph, flumetover, fluopicolide, fluopyram, 5 zoxamide, N-(3-ethyl-3,5,5-trimethyl-cyclohexyl)-3-formylamino-2-hydroxy-benzamide, carpropamid, dicyclomet, mandiproamid, oxytetracyclin, silthiofam and N-(6-methoxy-pyridin-3-yl) cyclopropanecarboxylic acid amide;

D) heterocyclic compounds, including 10 fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine, 2,3,5,6-tetra-chloro-4-methanesulfonyl-pyridine, 3,4,5-trichloropyridine-2,6-di-carbonitrile, N-(1-(5-bromo-3-chloro-pyridin-2-yl)-ethyl)-2,4-dichloro-nicotinamide, N-[(5-bromo-3-chloro-pyridin-2-yl)-methyl]-2,4-dichloro-nicotinamide, bupirimate, cyprodinil, diflumetorim, fenarimol, ferimzone, mepanipyrim, nitrapyrin. nuarimol, pyrimethanil, triforine, fenpiclonil, fluioxonil, aldimorph, dodemorph, dodemorph-acetate, fenpropimorph, tridemorph, fenpropidin, fluoroimid, iprodione, chlozolinate, procymidone, vinclozolin, famoxadone, fenamidone, fiutianll, octhilinone, probenazole, 5-amino-2-isopropyl-3-oxo-4-ortho-tolyl-2,3-dihydro-20 pyrazole-1-carbothioic acid S-allyl ester, acibenzolar-S-methyl, amisulbrom, anilazin, blasticidin-S, captafol, captan, chinomethionat, dazomet, debacarb, diclomezine, difenzoquat, difenzoquat-methylsulfate, fenoxanil, Folpet, oxolinic acid, piperalin, proquinazid, pyroquilon, quinoxyfen, triazoxide, tricyclazole, 2-butoxy-6-iodo-3-propylchromen4-one, 5-chloro-1-(4,6-dimethoxy-pyrimidin-2-yl)-2-methyl-1H-benzoimidazole, 5-chloro-7-(4-methylpiperidin-1-yl)-6-(2,4,6-trifluorophenyl)-[1,2,4]triazolo[1,5-a]pyrimidine and 5-ethyl-6-octyl-[1,2,4]triazolo[1,5-a]pyrimidine-7-ylamine;

E) carbamates including ferbam, mancozeb, maneb, metam, methasulphocarb, metiram, propineb, thiram, zineb, ziram, benthiavalicarb, pyributicarb, diethofencarb, iprovalicarb, iodocarb, propamocarb, propamocarb hydrochlorid, prothiocarb, valiphenal and N-(1-(1-(4-cyano-phenyl)ethanesulfonyl)-but-2-yl) carbamic acid-(4-fluorophenyl) ester.

Other active compounds, include guanidines: guanidine, dodine, dodine free base, guazatine, guazatine-acetate, iminoctadine, iminoctadine-tri acetate, iminoctadine-tris(albesilate); antibiotics: kasugamycin, kasugamycin hydrochloride-hydrate, streptomycin, polyoxine, validamycin A; nitrophenyl derivates: binapacryl, dinobuton, dinocap, meptyldinocap, nitrthal-isopropyl, tecnazen, organometal compounds: fentin salts, such as fentin-acetate, fentin chloride or fentin hydroxide; sulfur-containing heterocyclyl compounds: dithianon, isoprothiolane; organophosphorus compounds: edifenphos, fosetyl, fosetyl-aluminum, iprobenfos. phosphorous acid and its salts, pyrazophos, tolclofos-methyl; organochlorine compounds: chlorothalonil, dichlofluanid, dichlorophen, flusulfamide, hexachlorobenzene, pencycuron, pentachlorphenole and its salts, phthalide, quintozene, thiophanate-methyl, tolylfluanid, N-(4-chloro-2-nitro-phenyl)-N-ethyl-4-methyl-benzenesulfonamide; inorganic active substances: Bordeaux mixture, copper acetate, copper hydroxide, copper oxychloride, basic copper sulfate, sulfur; others: biphenyl, bronopol, cyflufenamid, chloroneb, cymoxanil, dicloran, tecnazene, diphenylamin, metrafenone, mildiomycin, oxin-copper, prohexadione-calcium, spiroxamine, tolylfluanid, N-(cyclopropylmethoxyimino-(6-difluoro-methoxy-2,3-difluoro-phenyl)-methyl)-2-phenyl acetamide, N′-(4-(4-chloro-3-trifluoromethyl-phenoxy)-2,5-dimethyl-phenyl)-N-ethyl-N-methyl formamidine, N′-(4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2-methyl-4-(3-tri-methylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl-piperidin-4-yl}-thiazole-4-carboxylic acid methyl-(R)-1,2,3,4-tetrahydro-naphthalen-1-yl-amide, acetic acid 6-tert-butyl-8-fluoro-2,3-dimethyl-quinolin-4-yl ester and methoxy-acetic acid 6-tert-butyl-8-fluoro-2,3-dimethyl-quinolin-4-yl ester;

Biological control is defined as the reduction of pest population by natural enemies and typically involves an active human role. The biological control of plant diseases is most often based on an antagonistic action of the BCA. There are several mechanisms by which biocontrol is thought to work, including the production of antibiotics, competition for nutrients and rhizosphere colonization.

Suitable biological control agents include non-pathogenic bacteria, including but not limited to Pseudomonas fluorescens, Pseudomonas putida, Streptomyces griseus, Streptomyces ochraceisleroticus, Streptomyces graminofaciens, Streptomyces corchousii, Streptomyces spiroverticillatus, Streptomyces griseovirdis, Streptomyces hygroscopicus, Bacillus subtilis, Bacillus cereus, Bacillus mycoides, Bacillus pumilus, Bacillus licheniformis, Bacillus thuringensis, and metabolites produced from said bacteria; non-pathogenic fungi are preferably selected from Trichoderma spp., Trichoderma harzianum, Trichoderma viridae, Verticillium lecanii, Sporidesmium sclerotiorum and Zygomycetes, and metabolites produced from said fungi; resin acids; plant extracts of Reynoutria sachallnensis; and plant defense induction agents, preferably harpin.

Examples of insecticides include:

A) pyrethroid compounds including acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin and transfluthrin;

B) nicotinic receptor agonists/antagonists compounds including acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thtocyclam, thiosultap-sodium and AKD1022;

C) GABA gated chloride channel antagonist compounds including chlordane, endosulfan, gamma-HCH (lindane); ethiprole, fipronil. pyrafluprole and pyriprole;

D) chloride channel activators including abamectin, emamectin benzoate, milbemectin and iepimectin; and

E) inhibitors of chitin biosynthesis including (e1) benzoyl ureas: bistrifluron, chlorfluazuron, diflubenzuron, flucycloxuron, flufenoxuron, hexaflumuron, lufenuron, novaluron, noviflumuron, teflubenzuron, triflumuron.

Examples of nematicides include: antibiotic nematicides, such as abamectin;vbotanical nematicides, such as carvacrol; extracts of Quillaja or Gleditsia; saponines; carbamate nematicides selected from benomyl, carbofuran, carbosulfan and cloethocarb; oxime carbamate nematicides selected from alanycarb, aldicarb, aldoxycarb, oxamyl and tirpate; fumigant nematicides selected from dithioether and methyl bromide; organophosphorus nematicides: organophosphate nematicides selected from diamidafos; fenamiphos; fosthietan and phosphamidon; organothiophosphate nematicides selected from cadusafos, chlorpyrifos, dichlofenthion, dimethoate, ethoprophos, fensulfothion, fosthiazate, heterophos, isamidofos, isazofos, phorate, phosphocarb, terbufos, thionazin and triazophos; phosphonothioate nematicides selected from imicyafos and mecarphon; and unclassified nematicides selected from acetoprole, benclothiaz, chloropicrin, dazomet, DBCP, DCIP, 1,2-dichloropropane, 1,3-dichloropropene, fluensulfone, furfural, metam, methyl iodide, methyl isothiocyanate and xylenols.

Specific examples of growth-promoting bacteria include bacteria of the genera azospirillum, azotobacter, azomonas, bacillus, beijerinckia, burkholderia, Clostridium, cyanobacteria, enterobacter, erwinia, gluconobacter, klebsiella and streptomyces, Azospirillum amazonense, Herbaspirillum seropedicae, Herbaspirillum rubrisubalbicans, Burkholderia tropica, Gluconacetobacter diazotrophicus, Pseudomonas fluorescens, Pseudomonas putida. Streptomyces griseus, Streptomyces ochraceisleroticus, Streptomyces graminofaciens, Streptomyces corchousii, Streptomyces spiroverticillatus. Streptomyces griseovirdis, Streptomyces hygroscopicus, Bacillus subtilis, Bacillus cereus, Bacillus mycoides, Bacillus pumilus, Bacillus licheniformis and Bacillus thuringensis.

Specific examples of growth regulators include acylcyclohexanediones, such as prohexadione, prohexaione-Ca, trinexapac or trinexapac ethyl; mepiquat chloride and chlormequatchloride.

Fertilizers are those customarily used in the cultivation of plants, particularly sugarcane plants, such as NPK fertilizers, ammonium sulfate, ammonium phosphate, ammonium nitrate, ureas, products of vegetable origin, such as cereal meal, tree bark meal, wood meal, nutshell meal and mulch, and mixtures thereof.

A superabsorber is a superabsorbent polymer having an absorption capacity for deionized water of least 100 g/1 g of polymer. Superabsorbent polymers are well-known synthetic organic polymers which are solid and hydrophilic, which are insoluble in water, and which are capable of absorbing a multiple of their weight of water or aqueous solutions, thereby forming a water containing polymer gel. They may be nonionic or ionic crosslinked polymers. Suitable superabsorbent polymers are for example known from U.S. Pat. No. 4,417,992, U.S. Pat. No. 3,669,103, International Patent Application Publication Nos. WO 01/25493 and WO 2008/031870. They are also commercially available, e.g. from SNF SA., France, under the trademark Aquasorb®, e.g. 3500 S, or from BASF SE under the trade names Luquasorb®, e.g. Luquasorb® 1010, Luquasorb® 1280, Luquasorb® 1060, Luquasorb® 1160, Luquasorb® 1061 and HySorb®.

### Definitions

As used in the specification and the appended claims, the singular forms “a,” “an” and “the” include plural referents unless the context clearly dictates otherwise. By way of example, “an element” means one or more element.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

In this specification and in the claims which follow, reference will be made to a number of terms which shall be defined to have the following meanings:

As used herein, the term “plant” includes any plant species, including, but not limited to, monocots (e.g., maize, sugarcane, wheat, rice, barley, sorghum, or rye) and dicots (e.g., soybean, Brassica, sunflower, cotton, or alfalfa). Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. A plant may be a transformed plant, wherein one or more heterologous or homologous nucleic acids reside in, or have been introduced into, the cells of the plant.

“Optional” or “optionally” means that the subsequently described event or circumstance may or may not occur, and that the description includes instances where said event or circumstance occurs and instances where it does not.

Though the invention described herein discusses sugarcane, the invention is not to be limited by this discussion, and it is contemplated that the methods and compositions disclosed herein are pertinent to other plant types disclosed and known to those of skill in the art.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

The disclosures provided herein may be embodied in many different forms and should not be construed as limited to the embodiments set forth herein. Like numbers refer to like elements throughout.

Many modifications and other embodiments of the inventions set forth herein will come to mind to one skilled in the art to which these inventions pertain having the benefit of the teachings presented in the foregoing descriptions and any associated drawings. Therefore, it is to be understood that the inventions disclosed herein are not to be limited to the specific embodiments disclosed and that modifications and other embodiments are intended to be included within the scope of the appended claims. Although specific terms are employed herein, they are used in a generic and descriptive sense only and not for purposes of limitation, unless otherwise defined.

### EXAMPLES

### Example 1

### Cultivation of Sugarcane Choplets, the Preparation of Plantlets and of Choplets

This Example describes the preparation of sugarcane choplets that can be used for encapsulation in a variety of containers including artificial seeds of sugarcane. Though the present examples are directed to sugarcane, it is to be understood that other types of plants could be used herein and the present invention contemplates use of other types of plants.

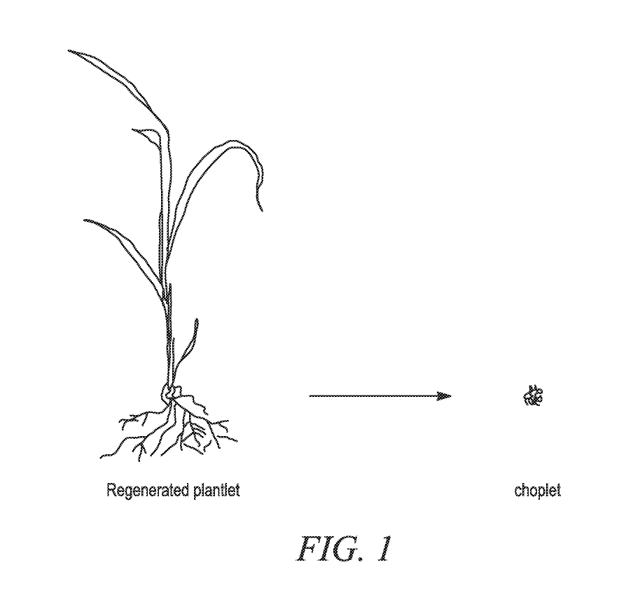

Choplets were prepared from plantlets that were propagated from sugarcane stalks of varieties CP01-1372 or KQ228, such varieties of sugarcane are described in International Publication No. WO2013096531. The plantlets could be produced in a variety of container types ranging from sterile 250 milliliters (mL) polycarbonate flasks with air filters to 1 liter (L) RITA™ bioreactors. The plantlets were propagated from fragments derived from proliferating bud tissue (e.g., explant tissue). The fragments were incubated in liquid plantlet regeneration medium (Murashige-Skoogs nutrient medium with 30 g/L sucrose and 0.2 weight percent Plant Preservative Mixture™ (PPM™) and 0.1 weight percent cefotaxime-hereinafter called LPM) for a period of at least ten days. The plantlets had leaves and shoots ranging from 1 cm to >10 cm. The plantlet was chopped using sterile scalpels or scissors to reduce the length of the shoots, leaves and roots of the plantlet to <1 mm in length extending from the base of the plantlet. What remained was a solid mass of tissue that has a volume as small as 1 mm³ and as large as 125 mm³. The sides of the mass ranged from 0.5 to 5 mm and the mass, herein referred to as a “choplet”, may be irregular in shape; and residual shoots, leaves and roots may be evident to the naked eye (FIG. 1), or shown in cross-section in FIG. 6 B and by laser tomography in FIGS. 7B and C. For example, a choplet of volume less than 27 mm³ may be used in an artificial seed, where the sides may be from 3 to 5 mm. A choplet is distinguished from embryonic or meristem tissue by microscopic analyses and by growth performance. See FIGS. 6 and 7. A choplet can develop into a plant comprising roots and shoots and/or leaves.

### Example 2

### Growth of Sugarcane Choplets in Artificial Environments

Following the preparation of choplets as described in Example 1, viability was measured by growth in LPM followed by transfer to soil and growth in a growth chamber or greenhouse. Approximately 30-50 choplets were transferred to a sterile Petri dish containing 5-10 mL of a liquid media which may be sterile water, LPM or 1/10th strength MS nutrients with 3 g/L sucrose, 0.2 wt % PPM, 0.1 wt % cefotaxime. The Petri dish was covered and sealed with a porous filter tape (Micropore™) to allow gas exchange and maintain sterility. The Petri dishes were placed on a gyrating rocker in a walk-in growth chamber where conditions were set for cycling between a 13 hour photoperiod at 150-220 microEinsteins/m²/s from Philips F32T8/ADV841/XEN 25 watt fluorescent tubes, with a relative humidity between 40 and 55 percent, temperature at 31° C. The 11 hour dark period temperature setting was at 22° C. The choplets were maintained in this setting until visible re-emergence of leaves or shoots. The time period ranged from hours to several days, for example an incubation period of 3 to 5 days was used. Table 1 describes typical germination frequencies following choplets of various regeneration age and incubation periods. Choplets that survived for 4 weeks typically had shoots of lengths greater than 5 cm and roots ≥1 cm in length.

After the incubation period, the choplets were transferred into soil medium for continued growth. Choplets were planted into flats containing an autoclaved synthetic soil such as commercial MetroMix™ 360 or a blended soil containing 49 weight percent sand, 49 weight percent Matapeake™ and 2 weight percent MetroMix™ 360. The soil was saturated with tap water before embedding the choplets into the soil. The choplets were oriented so that any emerging shoots and leaves were above the soil surface. The flats were covered with a transparent plastic dome lid and watered as needed from the bottom and top with water. Once a week, the flats were watered with a nutrient medium (either half-strength Hoagland's plant nutrient solution or a commercial all-purpose water-soluble household plant fertilizer such as Peters®). The progress of growth of the choplets into plantlets was monitored weekly. After 3 weeks, the clear dome was removed. The choplets continued to grow into plantlets and in several instances the plantlets were allowed to grow into full-grown sugarcane plants by successive transfer of the plants into larger pots when the plants became root-bound.

TABLE 1
Survival rates and average plant heights after 19 days in
MetroMix ™ soil in walk-in growth chambers

| Regenerated plantlet age (days | | |
|---|---|---|
| in liquid culture post-fragmentation) | Days in soil | % survival |
| 7 | 14 | <1 |
| 10 | 14 | 63 |
| | 28 | 48 |
| 17 | 14 | 18 |
| | 14 | 64 |
| | 14 | 92 |
| | 28 | 92 |

### Example 3

### Choplet Germination Yields when Pre-Incubating Choplets with Various Liquid Medium

This example describes choplet growth outcomes in soil (survival; morphology) when the plantlets were hardened by various treatments such as nutrient deprivation or osmoticum exposure. The hardening protocols for the plantlets were gradual (multi-day) acclimation of the tissue to decreasing sucrose and/or to increasing polyethylene glycol 6000 (PEG-6000) in LPM. The hardening treatment was evaluated at two stages: the growth of treated plantlets transplanted to synthetic soil; and the survival of choplets from plantlets in a controlled environment.

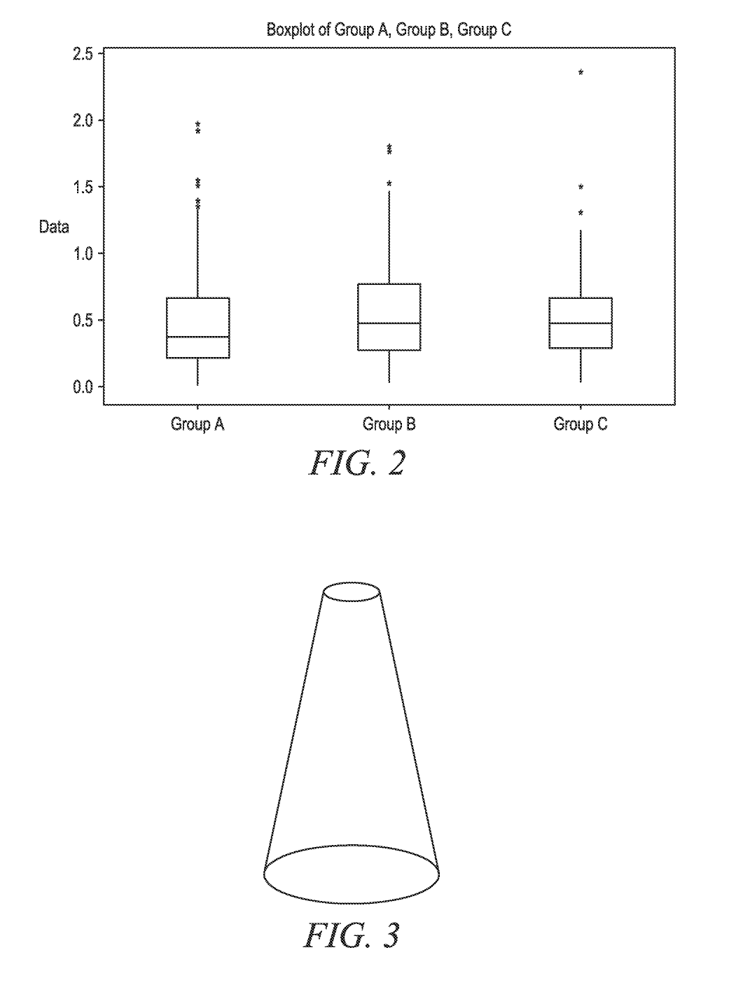

Three plantlet media were evaluated: Group A plantlets were grown in LPM, n=312 propagules; Group B plantlets were grown in LPM for 7 days followed by gradual step down in sucrose concentration every two days until a final sucrose concentration in LPM of 1.25%, n=312 propagules; Group C plantlets were grown in LPM for 7 days followed by transfer every two days to a medium containing lower sucrose levels and higher PEG-6000 levels until the medium content had 1.25% sucrose and 0.2% PEG-6000, n=216 propagules. At the end of growth in tissue culture (17 days), the resultant plantlet mean weights in the three treatment groups were similar (FIG. 2). When the treated plantlets were transplanted to soil, the combination of nutrient deprivation and osmoticum exposure showed an impact on plant growth and survival. Group C plantlets had lower survival rates and approximately 30% shorter heights than the plantlets in Groups A and B (Table 2).

TABLE 2
Survival rates and average heights of hardened plantlets
after 19 days in MetroMix ™ soil

| | Surviving plants in | | | |
|---|---|---|---|---|
| | artificial growth chamber | | | |
| | | alive @ | | Average shoot |
| Group | planted | day 19 | % surviving | height (cm) |
| A | 30 | 30 | 100 | 18 |
| B | 26 | 25 | 96 | 18 |
| C | 26 | 23 | 88 | 12 |

In another study, three hardening treatments were evaluated but group C hardening treatment was modified: Group C plantlets were grown in LPM for 7 days followed by transfer every two days to a medium containing higher PEG-6000 levels until the medium content had 3% sucrose and 0.2% PEG-6000. At the end of growth in tissue culture (17 days), the plantlets were transplanted to soil. The plant viability at days 10 and 17 are shown in Table 3. By decoupling the nutrient deprivation and osmoticum exposure, the repeated lower viability of the Group C plantlets shows that osmoticum treatment alone has a negative impact on plant growth and survival. In this study, the plantlets conditions with lowered sucrose had a lower viability than the controls.

The second test of these treated plantlets was to produce choplets and evaluate the choplets' success when transplanted to synthetic soil (Table 4). Choplets were produced from sugarcane plantlets followed by a one-day immersion in a media of water, a known plant media, or a known plant media or water containing a growth hormone, such as (6-benzylaminopurine (BAP) or gibberellins 3 (GA3)). Table 4 shows that the choplet survival rates in soil varies among the various subgroups. Choplet preparation may comprise plantlet hardening treatment and/or media treatment after formation of a choplet.

TABLE 3
Survival rates and average heights of hardened plantlets
after 19 days in MetroMix ™ soil

| | Surviving plants in | | | |
|---|---|---|---|---|
| | artificial growth chamber | | | |
| | | alive @ | Alive @ | % viable |
| Group | planted | day 10 | day 17 | (day 17) |
| A | 20 | 19 | 18 | 90 |
| B | 20 | 19 | 13 | 65 |
| C | 20 | 13 | 8 | 40 |

TABLE 4
Survival rates and average heights of choplets
from hardened plantlets after 10 day sin soil
Surviving plants in artificial growth chamber

| Hardening | 1-day hormone | | alive @ | |
|---|---|---|---|---|
| Group | choplet treatment | planted | day 10 | % surviving |
| A | Water | 40 | 17 | 43 |
| | BAP | 40 | 7 | 18 |
| | GA3 | 40 | 4 | 10 |
| B | Water | 40 | 7 | 18 |
| | BAP | 40 | 11 | 28 |
| | GA3 | 40 | 10 | 25 |
| C | Water | 40 | 17 | 43 |
| | BAP | 40 | 10 | 25 |
| | GA3 | 40 | 15 | 38 |

### Example 4

### Testing Survival and Growth of Choplets in Fields

In this Example, choplet survival in a cultivated field was influenced by several factors such as: field location; acclimation time post-choplet formation; and the presence of a protective seed. When choplets were allowed to harden in an artificial environment for times up to 10 days, the survival rate increased. The field was irrigated at least once a week to compensate for the absence of rain during the growing season. The duration of the field test ranged from 14 to 30 days. The nomenclature used for constructs: C or S describes the initial plant bioreactor treatment: C=control medium or LPM; S=LPM with 2.3 mg/L sodium silicate; plant size (L=large (plant leaf height >5 cm) M=medium (plant leaf height between 3 and 5 cm); or S=small (plant leaf height <3 cm)); seed design (CH, F4 or F7). Three field plantings during the short growing season in Delaware used a blend of plantlets of various sizes from RITA™ bioreactors and Plantforms™. The plantlets were used to prepare choplets, and the choplets were placed in sterile plastic Petri dishes with 5 mL of LPM. The Petri dishes were sealed with Micropore™ tape and placed on a rocker platform and incubated overnight in a walk-in growth chamber.

General steps of the seed assembly were as follows:

Seed structures identified as CH were 5 cm long paper tubes with tops constructed from microfuge tubes with the tube bottom cut to create a 5 mm diameter hole and with the lids removed (FIG. 3). The wider opening of this cone-like lid plastic tube was inserted into the top of the paper tube; the smaller opening was at the very top of the seed structure. The bottom of the structures were not sealed but closed by crimping. MetroMix™ soil (0.6-0.8 g) or an equivalent was added to the tube. The choplet was added and impressed into the top of the soil. Another 150 mg of soil was added and then an equivalent weight of tap water (circa 0.75 to 1 mL) was added. Water (1 mL) was added to wet the soil. The structure was tamped down to compact the soil, then covered with the lid.

Seed structures identified as F4 were constructed of 4 cm length waxed paper tubes. The top and bottoms of these F4 structures were an experimental film with a composition triblock polymer having a middle block that is poly(dimethylsiloxane) of number average molecular weight (Mₙ) and symmetric poly(D,L-lactide) end blocks (LDL). For example, see PCT/US2012/070766 (International Publication No. WO 2013/096531). The films were sealed to the edges of the tops and bottoms with a commercial quick sealing glue such as methyl-2- or ethyl-2-cyanoacrylate (KrazyGlue™). The F4 seed structure contents of soil, water and choplet was assembled as for CH.

Taller structures, labeled F7 were constructed of paper tubes of 7 cm length. The tops and bottoms were assembled exactly as described for F4. The seed structure contents contained 0.8 g of MetroMix™ soil initially and other contents were identical to that for F4.

The structures were usually assembled on the day before transfer to the farm for planting. Another test condition was that seed structures were assembled and acclimated in the growth chamber for 10 days and then were transported intact in small deep 24-well holding trays and transplanted to the field. The survival outcomes of these “acclimated” choplet artificial seeds varied by the block in the plot and are designated with the prefix “Acc”.

Planting #1. 347 choplet artificial seeds were constructed for immediate transfer and planting on the farm field; 28 “conditioned” choplet artificial seeds were transplanted. Choplets were generated in the 24 hour period prior to assembling the artificial seeds. 28 of 30 embedded choplets in 4 cm waxed paper tubes with LDL lids grew in the acclimated artificial growth chamber. Eight (8) of the 28 surviving artificial seeds emerged or broke through the top of the lid during this acclimation period. All 28 surviving artificial seeds were transported intact (all but one paper tube structure had enough physical integrity that they could be easily dug out of the flat in the growth chamber) in small deep 24-well holding trays and transplanted to one of 4 planting plots or blocks in the field.

Field outcomes: For the fresh choplet artificial seeds, the survival rate was six (6) percent: 21/347. Over eighty percent (80%) of the surviving choplets ( 17/21) were CH or the open polypropylene conical lids seed structures. Half of the 21 surviving plants from choplets were quite healthy but had not broken through the lid of the seed structure after 19 days in the open field. Higher survival numbers were seen for the acclimated choplet artificial seeds. By block, the survival numbers (and percentages) of these choplet artificial seeds: Block A: 6/7 (88%); Block C: 3/14 (21%); and Block D: 1/7 (14%). Overall, 36% of the Acc choplet artificial seeds survived and grew well through day 19 in the field. During the first few days after transplanting to the field, plants that had emerged and had exposed leaves exhibited signs of heat stress (browning leaves and deep brown leaf tips). Table 5 describes the plant morphology by blocks. Leaf heights were higher for the surviving plants from acclimated choplets; there were no significant differences in morphology among the surviving artificial seeds designs (Table 6).

TABLE 5
Leaf Height and Root length by Field Plot

| Field Block | Leaf Height (cm) | Root length (cm) |
|---|---|---|
| A | 6.87 ± 2.8 | |
| B | 6.52 ± 2.55 | 1.01 ± 0.5 |
| C | 5.33 ± 2.45 | |
| D | 6.11 ± 2.62 | 1.08 ± 0.84 |

TABLE 6
Leaf Height by Choplet seed structure design

| Structure- Field Block | Leaf Height (cm) |
|---|---|
| CH-A | 4.75 ± 2.24 |
| CH-C | 5.38 ± 2.46 |
| FH-C | 5.29 ± 2.63 |
| Acc-CH-A | 8.41 ± 2.11 |

Planting #2: A longer choplet artificial seeds “conditioning” in the growth chamber resulted in a significant improvement in field survival (compare 24% for fresh choplet artificial seeds versus 71% for acclimated choplet artificial seeds). 209 Artificial seeds containing choplets were assembled from 1 cm wide waxed paper tubes with either F4 or CH designs: 162 were fresh choplets; 47 were acclimated CH choplet artificial seeds. Overall, 24% of the choplets survived after 21 days in the field. The survival numbers were independent of lid structure—F4: 13 of 53 survived; CH: 27 of 109 survived. For acclimated choplet artificial seeds, 34 of 47 survived

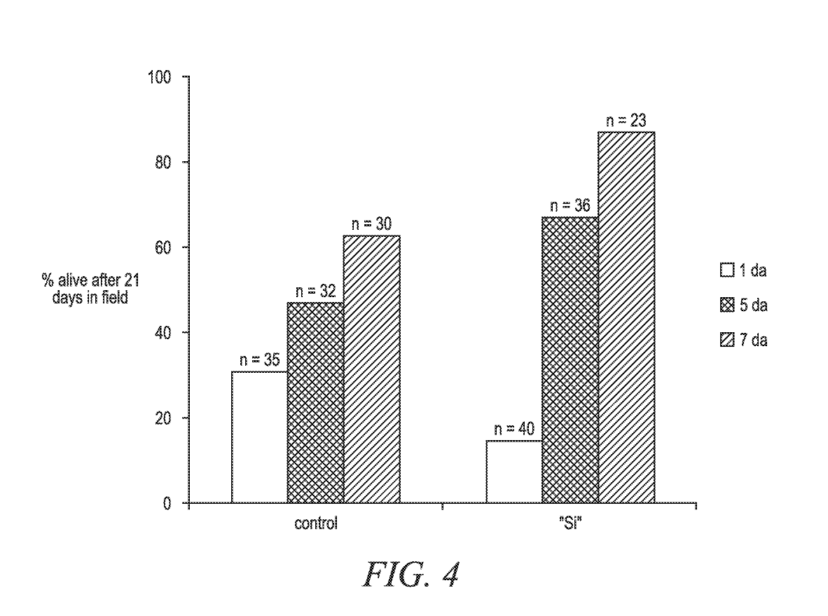

Planting #3: Choplets were acclimated for either 1, 5 or 7 days before constructing the artificial seeds in 1 cm diameter waxed paper tubes with crimped bottoms and open polypropylene cone-top lids. The choplet artificial seeds were transplanted and allowed to grow in the field for 21 days. Longer hardening time was beneficial to the field survival of choplets derived from LPM regenerated plantlets and from silicate-hardened regenerated plantlets (FIG. 4).

### Example 5

### Effect of Altering Shoot Trimming of Choplets

This example describes the survival effect of retaining longer choplets shoots. Typically, there was no significant visible change in root development during the 7-day conditioning period. A choplet with a larger shoot length (ca 1-2 cm) was compared with choplets where the shoots were nearly completely removed. The plantlets used for preparing choplets were from two treatment groups: (a) silicated with Paclobutrazol and giberellic acid; and (b) paclobutrazol and giberellic acid. The entire group of plantlets, regardless of size, were used to form choplets and were placed in sterile plastic Petri dishes to which ca. 5-7 mL of LPM was added to immerse a layer of choplets. Half of the plantlets in each treatment group were cut so that the choplet had shoots about 0.5 cm length. This choplet treatments was designated as “crewcuts” and differentiated from the standard choplet preparation in which substantially all shoot extension are removed, and were designated as “bald” choplets. A single artificial seed design—CH—(5 cm length waxed paper tube (1 cm diameter) with crimped bottom and polypropylene cone top lid) was used for this experiment.

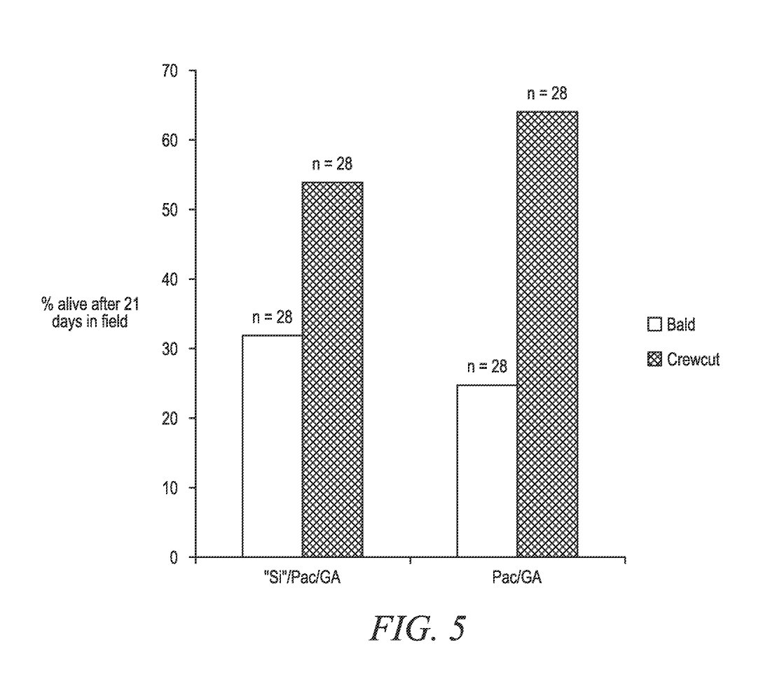

Artificial seeds were assembled with these choplets into CH waxed paper tubes the day before transplanting in the farm field with (n=75 total per treatment). Choplets having a short shoot tuft at day 1 conferred a clear advantage (crewcut choplets) over the bald choplets for field survival (FIG. 5) in both the control group and the silicate-treated plants.

### Example 6

### Survival Outcomes after Enclosing Choplets in Paper Tube Artificial Seeds

This example describes the soil growth advantage conferred by the enclosure or encapsulation of sugarcane choplets in paper tubes with Parafilm™ covers at bottom and top. The choplets were from plantlets produced by a standard plantlet production process or from a hardening treatment (e.g. high CO₂) in the temporary immersion Adaptis™ bioreactors. The choplets were incubated in various media. The media were sterile water; LPM; BAP=Benzyl adenylpurine prepared by solubilizing solid in dimethylsulfoxide to make 1 mg/mL stock then diluted stock solution in water (50 uL to 50 mL final volume) to prepare 1 mg/L working solution; GA3=giberellin 3 prepared by diluting commercial stock solution (1 mg/mL) in water to form a 1 mg/L working solution. The choplets were segregated into 4 groups of at least 50 choplets. Each group of choplets was weighed on a petri dish before addition of the 7 mL of bathing solution: water; LPM; BAP; and GA3. Each dish was covered and the edge was sealed with 3M Micropore™ tape. The petri dishes were secured to a nutator inside a walk-in growth chamber for 21 hr. For each choplet group, either ten (10) or twenty (20) F4 paper seed structures containing MetroMix™ soil and 200 uL water were assembled with triblock top and bottom lids. The structures were transplanted in a flat containing water-saturated MetroMix™ soil such that the top of the seed design is flush with the top of the soil surface. The survival outcomes of the unembedded choplets and the choplet artificial seeds are shown in Table 7. Encapsulating the choplet in an artificial seed provided improvement in soil survival for all media incubation groups except GA3.

TABLE 7
Soil outcomes of unembedded and embedded treated choplets

| | | | | Surviving |
|---|---|---|---|---|
| | Paper tube | Number | Surviving | on day 17 |
| Treatment | artificial seeds | Planted | on day 6 | (% surviving) |
| Water | | 18 | 10 | 1 (5.6) |
| LPM | | 15 | 14 | 8 (53) |
| BAP | | 14 | 6 | 1 (7) |
| GAS | | 14 | 9 | 4 (29) |
| Water | F4 | 10 | 10 | 4 (40) |
| LPM | F4 | 10 | 10 | 9 (90) |
| BAP | F4 | 20 | 20 | 11 (55) |
| GAS | F4 | 20 | 20 | 6 (30) |

In another study, slightly larger in volume choplets were prepared from medium, small and large plantlets that were grown with 2.3 mL/L sodium silicate treatment. These choplets had 2-4 millimeters of leaf and root tissue visible. These larger volume choplets were placed in a sterile petri dish containing 10 mL liquid plant medium (MS/3% sucrose, 0.2% PPM™), the petri dish was covered and side was sealed with Micropore® tape. After 1 day in walk-in chamber on a gently oscillating surface, a nutator, overnight, half of the choplets looked healthy with ca 3-5 mm emerged green shoot growth. The choplets were transferred into 4 cm paper tube structures with top and bottom triblock lids; the seed structure planting depth had 2 cm above the soil surface. Larger choplets embedded in a seed structure had high survival percent—ca. 90% (Table 8). The seed structure provided a good moisture barrier and a humidified atmosphere. The 26 choplet seed structures that survived this experiment were transplanted to the farm field.

TABLE 8
Plant Growth in seed structure:

| Day | Number of Structures |
|---|---|
| (number) | with Healthy Plant Tissue |
| 1 | 30 |
| 6 | 25 |
| 10 | 27 |
| 13 | 26 |

### Example 7

### Choplet Outcomes by Enclosing in Degradable, Sealed Artificial Seeds

This example demonstrates that choplets can be encapsulated in degradable sealed artificial seeds.

Choplets were packaged in bilayer cold-water soluble polyvinyl alcohol and rapeseed/Kraton® gel films and monolayer (rapeseed/Kraton® gel only) films—each artificial seed had ˜200 uL water added before heat sealing the artificial seedshut. In this example, 15 packaged choplets were planted below the surface of MetroMix™ soil and 5 artificial seeds comprising choplets were placed in an open Petri dish and served as a control. The artificial seeds were watered as needed. During the first week, the control samples (no soil) appeared healthy, but in the succeeding week, the tissue within these artificial seeds did not appear to thrive as well as their soil-sown counterparts which appeared greener. Shoots occasionally emerged and penetrated through the highly elastic film after 12 days in soil. The growth within these artificial seeds was slow compared to the choplets described in previous Examples.

That which is claimed:

1. A method of making a choplet, comprising,
a) removing from a plantlet that was grown for at least ten days under in vitro culture conditions at least a portion of the length of sprouted roots and shoots to form a choplet.

2. The method of claim 1, further comprising severing the choplet into smaller volume choplets.

3. The method of claim 2, further comprising, after severing the choplet into smaller volume choplets, placing the choplets into a liquid medium for a time period of 30 minutes to 10 days.

4. The method of claim 1, wherein the plantlet is produced by growing a plant tissue source material in a container for a predetermined time.

5. The method of claim 1, wherein the plantlet is severed to form choplets having a particular size sides and volume.

6. The method of claim 1, wherein the choplets have a volume of from about 0.5 mm³ to about 125 mm³.

7. The method of claim 1, wherein the choplet comprises a shoot apical meristem-like activity zone that is within about 0.1 mm to about 5 mm of a root apical meristem-like activity zone.

8. The method of claim 1, wherein the plantlet grows under in vitro culture conditions for 10 days.

9. The method of claim 1, wherein the plantlet grows under in vitro culture conditions for 14 days.

10. The method of claim 1, wherein the plantlet grows under in vitro culture conditions for 21 days.

11. The method of claim 2, wherein the liquid medium comprises water and optionally, compounds such as hormones, minerals, sugars, growth hormones, and hardening agents.

12. The method of claim 3, wherein the choplet resides in the liquid medium for a time period of about 30 minutes to about 7 days.

13. The method of claim 1, wherein the plantlet develops from a plant tissue source material that is meristematic tissue.

14. The method of claim 1, wherein the plantlet develops from a plant tissue source material that is a choplet.

15. The method of claim 3 wherein the liquid medium comprises water and optionally, supplements such as minerals, sugars, growth hormones, and hardening agents.

16. The method of claim 1, wherein the plantlet is selected from the group consisting of: sugarcane, Saccharum spp, Miscanthus, switchgrass, potato, banana, orchids, cocoa, and pine trees; b) a genetically modified plant of a), c) a micropropagated version of a), and d) a genetically modified, micropropagated version of a).

17. The method of claim 1, wherein the plantlet is sugarcane.

18. A choplet made by the method of claim 1.

19. The choplet of claim 18, wherein the shoot apical meristem-like activity zone is within about 0.01 mm to about 20 mm of the root apical meristem-like activity zone.

20. An artificial seed comprising the choplet of claim 1.

21. The artificial seed of claim 20, further comprising a structure suitable for planting.

22. A method for regenerating a plant, comprising, placing a choplet in a structure suitable for planting.

23. The method of claim 22, wherein the structure suitable for planting is a paper tube with a closure.

24. A method for regenerating a plant, comprising, placing a choplet in a field.
